(19) United States
(12) Plant Patent Application Publication
(10) Pub. No.: US 2016/0014947 P1
(43) Pub. Date: Jan. 14, 2016

(54) Dianthus plant named 'KLEDP14216'

(50) ;

(71) Applicant: Klemm+Sohn GmbH & Co. KG, Stuttgart (DE)

(72) Inventors: Nils Klemm, Stuttgart (DE); Martin Glawe, Stuttgart (DE)

(73) Assignee: Klemm+Sohn GmbH & Co. KG, Stuttgart (DE)

(21) Appl. No.: 14/545,535

(22) Filed: May 18, 2015

Related U.S. Application Data
(60) Provisional application No. 61/998,890, filed on Jul. 10, 2014.

(51) Int. Cl. A01H 5/00

(52) U.S. Cl. PLT/278

(57) ABSTRACT

A new variety of Dianthus designated ‘KLEDP14216’ having purple and dark-red double flowers and a mainly upright plant habit with little drooping, is disclosed.

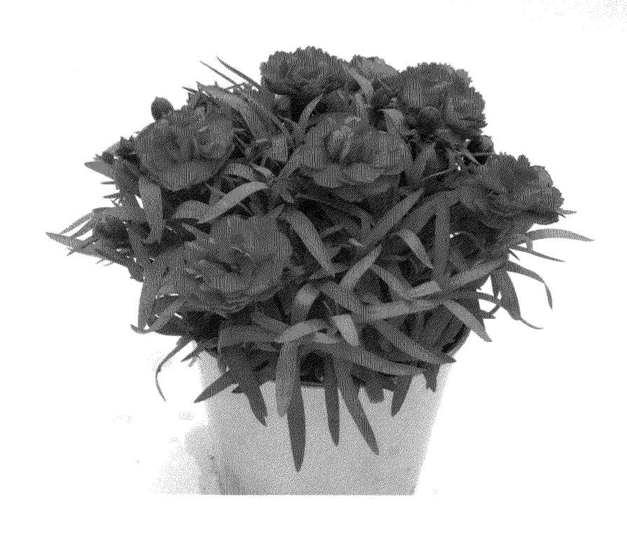

### CROSS-REFERENCE TO RELATED APPLICATIONS

This application claims the benefit of priority of U.S. Provisional Application No. 61/998,890 filed on Jul. 10, 2014 which is incorporated by reference herein in its entirety.

### GENUS AND SPECIES

Dianthus caryophyllus

### VARIETY DENOMINATION

‘KLEDP14216’

### BACKGROUND

The present invention comprises a new and distinct variety of Dianthus, botanically known as Dianthus caryophyllus, and hereinafter referred to by the variety name ‘KLEDP14216’. ‘KLEDP14216’ originated from a cross-pollination between the proprietary female Dianthus parent ‘DCP 2006 0122’ (unpatented) and the proprietary male Dianthus parent ‘DCP 2007 0092’ (unpatented) in May 2008 in Stuttgart, Germany.

The seeds from the cross were sown and plants were grown in a greenhouse for evaluation, where an individual plant designated ‘KLEDP14216’ was selected from the group of plants in May 2009 in Stuttgart, Germany. In October2009, ‘KLEDP14216’ was first vegetatively propagated by vegetative cuttings. ‘KLEDP14216’ was found to reproduce true to type in successive generations of asexual propagation in Stuttgart, Germany via vegetative cuttings.

### SUMMARY

The following are the most outstanding and distinguishing characteristics of this new variety when grown under normal horticultural practices in Stuttgart, Germany.

1. Purple and dark-red double flowers; and

2. A little hanging character but first upright, stems and flower stems are more hanging from beginning of the flowering.

### DESCRIPTION OF THE PHOTOGRAPH

This new Dianthus plant is illustrated by the accompanying photograph of a plant approximately 10 to 12-weeks old and grown in a glass greenhouse in Stuttgart, Germany. The colors shown are as true as can be reasonably obtained by conventional photographic procedures. The photograph shows the whole plant habit, including flowers, foliage, and buds of ‘KLEDP14216’ taken in April 2012.

### DESCRIPTION

The following detailed descriptions set forth the distinctive characteristics of ‘KLEDP14216’. The data which define these characteristics were collected from asexual reproductions carried out in Stuttgart, Germany. Data was collected on 5-month old plants grown in a greenhouse in May 2014. Color references are to The R.H.S. Colour Chart of The Royal Horticultural Society of London (R.H.S.), 5^{th} edition (2007).
Classification:
   Family.—Caryophyllaceae.
   Botanical.—Dianthus caryophyllus.
   Common.—Pot carnation.
   Designation.—‘KLEDP14216’.
Parentage:
   Female parent.—The proprietary Dianthus variety ‘DCP 2006 0122’ (unpatented).
   Male parent.—The proprietary Dianthus variety ‘DCP 2007 0092’ (unpatented).
Plant:
   Propagation type.—Vegetative cuttings.
   Time to produce a rooted young plant.—14 to 16 days.
   Root description.—White and fine.
   Rooting habit.—Lanceolate.
   Plant form.—Potted and hanging plant.
   Plant height.—10.0 cm to 12.0 cm.
   Plant diameter or spread.—25.0 cm to 27.0 cm.
Lateral branches:
   Length.—8.0 cm to 10.0 cm.
   Diameter.—3.0 cm to 5.0 cm.
   Internode length.—1.0 cm.
   Node length.—0.2 cm.
   Node diameter.—0.2 cm.
   Texture.—Smooth.
   Color.—RHS N137B.
Foliage:
   Arrangement.—Opposite.
   Length.—4.5 cm to 5.5 cm.
   Width.—0.7 cm to 0.9 cm.
   Shape.—Lanceolate.
   Apex.—Acute.
   Base.—Obtuse.
   Margin.—Entire.
   Texture (both upper and lower surfaces).—Smooth.
   Immature leaf color.—Upper surface: RHS 137B Lower surface: RHS 137B.
   Mature leaf color.—Upper surface: RHS N137A Lower surface: RHS N137D.
   Venation pattern.—Palmate.
   Venation color (both upper and lower surfaces).—RHS N137A.
Flowers:
   Flower type and habit.—Double, round, small spray.
   Fragrance.—Absent.
   Natural flowering season.—Early spring to fall.
   Flower longevity on the plant.—10 to 12 days.
   Buds.—Length: 1.3 cm to 1.5 cm Diameter: 0.8 cm to 1.0 cm Shape: Ovate Color: RHS 187A.
   Corolla.—Quantity of petals and arrangement: Greater than 30 and arranged in several whorls Corolla height: 1.6 cm to 2.0 cm Petal length: 4.0 cm Petal width: 2.0 cm Petal shape: Spoon-shaped Petal apex: Obtuse Petal base: Acute Petal margin: Crenate Petal texture (both upper and lower surfaces): Satiny Petal color: Immature: Upper surface: RHS 46B Lower surface: RHS 185B Mature: Upper surface: RHS 46A Lower surface: RHS 185A.
   Calyx.—Quantity of sepals: Composed of 4 to 5 fused sepals Calyx height: 1.4 cm to 1.6 cm Calyx diameter (at the apex): 0.6 cm to 0.8 cm Sepal texture (both upper and lower surfaces): Smooth Sepal color: Upper surface: RHS N77A Lower surface: RHS 137B.
Peduncles:
   Length.—10.0 cm to 12.0 cm.
   Diameter.—0.2 cm.
   Strength.—Weak.
   Texture.—Smooth.
   Color.—RHS N137A.
Reproductive organs:
   Stamens.—Quantity per flower: 3 to 5 Filament length: 0.4 cm to 0.6 cm Filament color: RHS 155A Anther length: 0.1 cm Anther diameter: 0.05 cm Anther color: RHS 26D Pollen amount: Few.
   Pistils.—Quantity per flower: 1 Pistil length: 1.5 cm to 1.7 cm Style length: 0.1 cm Style color: RHS N155B Stigma shape: Filamentous Stigma color: RHS 187A Ovary size: 0.7 cm to 0.9 cm Ovary shape: Ovate Ovary color: RHS 145A.
Fruit and seed set: No fruit and seed set observed.
Disease and pest/insect resistance: No disease and pest/insect resistance observed.

### COMPARISON WITH PARENTAL LINES AND KNOWN VARIETY

‘KLEDG14216’ is a distinct variety of Dianthus and is distinguished from its parents as described in Table 1.

TABLE 1
Comparison with Parental Lines

| | | Female Parent | Male Parent |
|---|---|---|---|
| Characteristic | ′KLEDP14216′ | ′DCP 2006 0122′ | ′DCP 2007 0092′ |
| Flower color | Purple and dark- | Dark-red | Purple |
| | red | | |
| Plant habit | Little drooping | Upright | Drooping |

‘KLEDP14216’ is a distinct variety of Dianthus and is most similar to the commercial Dianthus variety ‘Rubin’ (patent status unknown). Differences between the varieties are described in Table 2.

TABLE 2
Comparison with Similar Variety

| Characteristic | ′KLEDP14216′ | ′Rubin′ |
|---|---|---|
| Plant habit | Little drooping | Upright |
| Flower type | Double | Semi-double |

We claim:

1. A new and distinct variety of Dianthus plant designated ‘KLEDP14216’ as illustrated and described herein.
